(19) United States
(12) Patent Application Publication
(10) Pub. No.: US 2025/0253367 A1
(43) Pub. Date: Aug. 7, 2025

(54) Methods, Devices, Controllers, and Media for Determining Fuel Cell Anode Water Condition

(71) Applicant: Robert Bosch GmbH, Stuttgart (DE)

(72) Inventors: Huixiang Zhang, Wuxi (CN); Chengjun Tao, Wuxi (CN); Dongge Chen, Wuxi (CN); Ren Dong, Wuxi (CN); Weiwei Tao, Wuxi (CN); Xianghua Yuan, Wuxi (CN)

(21) Appl. No.: 19/038,157

(22) Filed: Jan. 27, 2025

(30) Foreign Application Priority Data
Feb. 1, 2024 (CN) 2024 1014 8320.5

(51) Int. Cl. H01M 8/04492; H01M 8/0438; H01M 8/04537; H01M 8/04955

(52) U.S. Cl. CPC H01M 8/045; H01M 8/04388; H01M 8/0441; H01M 8/04589; H01M 8/04649; H01M 8/04955

(57) ABSTRACT

Methods, devices, controllers, vehicles, and media for determining the anode water condition of a fuel cell system are disclosed. The method includes (i) determining the anode inlet humidity of the fuel cell system, (ii) determining the separation efficiency of a water-gas separator of the fuel cell system, and (iii) determining the condition of the water at the anode of the fuel cell system based on the anode inlet humidity and the separation efficiency of the water-gas separator. In this way, the possibility of flooding at the anode can be determined in advance of the damage to the stack, thereby enabling timely application of appropriate protective measures to reduce the damage to the stack from the flooding.

This application claims priority under 35 U.S.C. § 119 to patent application no. CN 2024 1014 8320.5, filed on Feb. 1, 2024 in China, the disclosure of which is incorporated herein by reference in its entirety.

The present disclosure relates to the field of fuel cell systems and more particularly to methods, devices, controllers, vehicles, and media for determining fuel cell anode water condition.

### BACKGROUND

A fuel cell system is a device that generates electrical energy through a chemical reaction of hydrogen with oxygen. In the chemical reaction, hydrogen and oxygen are combined to generate water and the generated water is one of the main by-products of the generation of the electrical energy. The water vapor and liquid water generated in the fuel cell system need to be expelled. If excessive liquid water is accumulated at the anode of the stack, the anode will be flooded, which can lead to a degradation of system performance and even damage to the system. A water-gas separator is located at the rear end of the stack of the fuel cell system and is used to separate water mixed in gases such as hydrogen discharged at the anode outlet from the gas. In order to avoid liquid water entrainment, the water-gas separator is required to have a strong water-gas separation efficiency.

The water that is captured by the water-gas separator may be controlled by a drain valve located at the rear end of the water-gas separator. When the drain valve is opened, the water may flow to a drain device, thereby being drained out of a vehicle; and when the drain valve is closed, the water may be stored in the water-gas separator. The drain valve plays an important role in protecting an apparatus, maintaining system performance, and discharging moisture generated in the fuel cell system, thereby ensuring that the system is capable of operating stably and generating the required electrical energy.

### SUMMARY

Examples of the present disclosure present methods, devices, controllers, vehicles, and media for determining fuel cell anode water condition. In the examples of the present disclosure, the humidity at the anode inlet of the fuel cell system as well as the separation efficiency of the water-gas separator can be monitored. Whether flooding will occur at the anode may then be determined based on the humidity at the anode inlet and the separation efficiency of the water-gas separator. In this way, the possibility of flooding at the anode can be determined in advance of the damage to the stack, thereby enabling timely application of appropriate protective measures to reduce the damage to the stack from the flooding. In addition, using the humidity at the anode inlet and the separation efficiency of the water-gas separator to predict flooding can reduce additional sensors and hardware equipment, thereby reducing costs and improving system robustness.

In a first aspect of the present disclosure, a method for determining the water condition at the anode of a fuel cell system is provided. The method comprises determining the anode inlet humidity of the fuel cell system. The method further comprises determining the separation efficiency of the water-gas separator of the fuel cell system. Additionally, the method further comprises determining the condition of the water at the anode of the fuel cell system based on the anode inlet humidity and the separation efficiency of the water-gas separator.

In a second aspect of the present disclosure, a device is provided. The device comprises a humidity determination unit configured to determine the anode inlet humidity of the fuel cell system. The device further comprises a separation efficiency determination unit configured to determine the separation efficiency of the water-gas separator of the fuel cell system. Additionally, the device further comprises a flooding monitoring unit configured to determine the condition of the water at the anode of the fuel cell system based on the anode inlet humidity and the separation efficiency of the water-gas separator.

In a third aspect of the present disclosure, a controller is provided. The controller comprises one or more processors; and a storage device for storing one or more programs, which, when the one or more programs are executed by the one or more processors, cause the one or more processors to implement a method for determining the water condition at the anode of the fuel cell system. The method comprises determining the anode inlet humidity of the fuel cell system. The method further comprises determining the separation efficiency of the water-gas separator of the fuel cell system. Additionally, the method further comprises determining the condition of the water at the anode of the fuel cell system based on the anode inlet humidity and the separation efficiency of the water-gas separator.

According to a fourth aspect of the present disclosure, a vehicle is provided. The vehicle includes a controller provided according to the third aspect of the present disclosure.

In a fourth aspect of the present disclosure, a computer-readable storage medium is provided. The computer-readable storage medium has computer-executable instructions stored thereon, wherein the computer-executable instructions are executed by a processor to implement the method provided according to the first aspect of the present disclosure. It will be understood that the content described in the Summary is not intended to limit key or important features of the examples of the present disclosure, nor is it intended to limit the scope of the present disclosure. Other features of the present disclosure will become readily understood by the following description.

### BRIEF DESCRIPTION OF THE DRAWINGS

Above and other features, advantages and aspects of various examples of the present disclosure will become more apparent in combination with the accompanying drawings and with reference to the following detailed description. In the accompanying drawings, like or similar accompanying drawings designate like or similar elements, wherein:

FIG. 1 shows a schematic diagram of an example environment in which a plurality of examples of the present disclosure may be implemented;

FIG. 2 shows a flow chart of a method for determining the water condition at a fuel cell system anode according to some examples of the present disclosure;

FIG. 3 shows a schematic diagram of an example of determining that flooding will occur at the anode based on anode inlet humidity and the separation efficiency of the water-gas separator according to some examples of the present disclosure;

FIG. 4 shows a schematic diagram of an example of controlling a drain valve based on anode inlet humidity and the separation efficiency of the water-gas separator according to some examples of the present disclosure;

FIG. 5 shows a schematic diagram of an example of determining that flooding will occur at the anode based on anode inlet humidity, the separation efficiency of the water-gas separator, and the high-frequency impedance of the stack according to some examples of the present disclosure;

FIG. 6 shows a schematic diagram of an example in which the state of a stack as indicated by the anode inlet humidity and the high-frequency impedance of the stack is inconsistent according to some examples of the present disclosure;

FIG. 7 shows a schematic diagram of an example of determining a stack is membrane-dry based on the anode inlet humidity and the high-frequency impedance of the stack according to some examples of the present disclosure;

FIG. 8 shows a schematic diagram of an example of using a virtual humidity sensor to determine anode inlet humidity according to some examples of the present disclosure;

FIG. 9 shows a schematic diagram of an example of using a separation efficiency determination model to determine the separation efficiency of the water-gas separator according to some examples of the present disclosure;

FIG. 10 shows a block diagram of a device for determining the water condition at a fuel cell system anode according to some examples of the present disclosure;

FIG. 11 shows a block diagram of an apparatus that can implement a plurality of examples of the present disclosure.

### DETAILED DESCRIPTION

The examples of the present disclosure will be described in further detail below with reference to the accompanying drawings. While certain examples of the present disclosure are shown in the accompanying drawings, it should be understood that the present disclosure may be implemented in various forms and should not be construed as being limited to the examples set forth herein, rather these examples are provided for a more thorough and complete understanding of the present disclosure. It will be understood that the accompanying drawings and examples of the present disclosure are for exemplary purposes only and are not intended to limit the scope of protection of the present disclosure, and the examples of the present disclosure that are described below with reference to the accompanying drawings are for illustrative purposes only.

A fuel cell system generates electrical energy through a chemical reaction of hydrogen with oxygen. The fuel cell system comprises an anode and a cathode, hydrogen may be introduced at the anode, and oxygen may be introduced at the cathode. A proton exchange membrane is a semi-permeable membrane located between the anode and the cathode, which is used to conduct protons and isolate reactants located at the anode and the cathode. In an electrochemical reaction, the hydrogen at the anode binds to the oxygen at the cathode, water is generated at the cathode, and the generated water is one of the main byproducts of the generation of electrical energy. However, part of water at the cathode may permeate to the anode through a proton exchange membrane, so there is a need to discharge the water at the anode in time to prevent damage to the stack due to water inundation at the anode.

In some conventional solutions, cell voltage detectors (CVMs) may be utilized to monitor if the stack is flooded. In general, the cathode of the fuel cell is the positive electrode of the battery, the anode is the negative electrode of the battery, and the voltage of the cathode is higher than the voltage of the anode. However, excessive liquid water at the anode (i.e., flooding) may result in the anode voltage being higher than the cathode voltage, thereby creating a reverse voltage. Conventional solutions can utilize CVMs to collect and transmit voltage from a monolithic battery to the fuel cell system control unit and then determine if the stack is flooded by checking the monolithic voltage of the stack. For example, if the control unit finds that the monolithic voltage is a reverse voltage, it may be determined that the stack has been flooded. However, in these conventional solutions, the reverse voltage can only be detected when the stack has already been flooded. The stack may have already been damaged despite immediate countermeasures at this time. In addition, the cost of CVMs is high, and the use of CVMs to monitor if a stack is flooded increases the manufacturing costs of the vehicle.

To this end, examples of the present disclosure present a scheme for determining the water condition at the anode of the fuel cell system. In the examples of the present disclosure, the humidity at the anode inlet of the fuel cell system as well as the separation efficiency of the water-gas separator can be monitored. Whether flooding will occur at the anode may then be determined based on the humidity at the anode inlet and the separation efficiency of the water-gas separator. In this way, the possibility of flooding at the anode can be determined in advance of the damage to the stack, thereby enabling timely application of appropriate protective measures to reduce the damage to the stack from the flooding. In addition, using the humidity at the anode inlet and the separation efficiency of the water-gas separator to predict flooding can reduce additional sensors and hardware equipment, thereby reducing costs and improving system robustness.

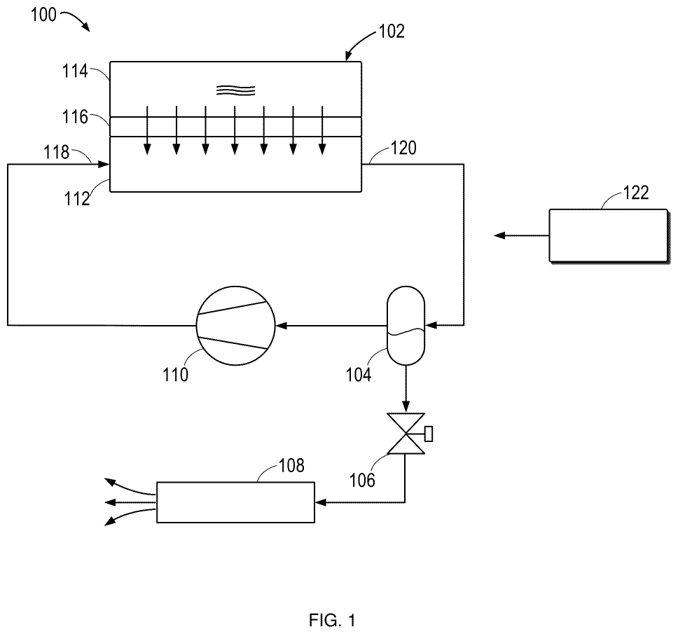

FIG. 1 shows a schematic diagram of an example environment 100 in which a plurality of typical examples of the present disclosure may be implemented. As shown in FIG. 1, the environment 100 comprises a stack 102, a water-gas separator 104, a drain valve 106, an exhaust device 108, and a hydrogen circulating pump 110. The stack 102 comprises an anode 112, a cathode 114, and a proton exchange membrane 116. In the environment 100 as shown, hydrogen enters the stack 102 from the inlet 118 of the anode 112, the hydrogen electrochemically reacts with the oxygen at the cathode 114, and water is generated at the cathode 114. Typically, most of the generated water may be discharged out of the cathode 114 through an air outlet; however, because the water concentration at the cathode 114 is higher, a water concentration difference is formed on both sides of the proton exchange membrane 116, so that part of water (also known as permeate water) diffuses to the anode 112 through the proton exchange membrane 116. When the permeate water is expelled from the outlet 120 of the anode 112, it may enter and be captured by the water-gas separator 104.

However, since the separation efficiency of the water-gas separator 104 is generally not 100%, water not captured by the water-gas separator 104 is recycled back to the inlet 118 of the anode 112 of the stack 102 under the action of the hydrogen circulating pump 110. Accordingly, the humidity at the inlet 118 of the anode 112 may reflect to some extent whether liquid water has accumulated at the anode 112, but it cannot reflect whether the accumulated liquid water exceeds the range of normal operation of the stack 102. For example, when the humidity at the inlet 118 of the anode 112 reaches a higher value (e.g., 95%, 98%, or 100%), it may be inferred that there is a higher likelihood that the anode 112 has accumulated liquid water, but it is difficult to deduce from that humidity value whether the amount of liquid water accumulated is large or small.

As shown in FIG. 1, in the environment 100, the water that is successfully captured by the water-gas separator 104 may be stored in the water-gas separator 104, and the water-gas separator 104 is connected or integrated with the electrically controlled drain valve 106. The frequency and duration of opening the drain valve 106 may be controlled by a control unit 122 of the fuel cell system. When the drain valve 106 is opened, the water stored in the water-gas separator 104 may flow to the exhaust device 108 and be discharged out of the vehicle by the exhaust device 108. In this way, the water at the anode 112 may be reduced by opening the drain valve 106. However, since the water-gas separator 104 is incapable of completely separating hydrogen from water, part of the hydrogen will be discharged out of the vehicle along with water when the drain valve 106 is opened. If the opening frequency of the drain valve 106 is too high or the duration is too long, it may cause hydrogen to increase as the water is discharged out of the vehicle, thereby violating safety standards. Thus, the flooding of the stack 102 cannot be prevented simply by frequently opening the drain valve 106.

In some examples of the present disclosure, the control unit 122 may determine the humidity at the inlet 118 of the anode 112 and the separation efficiency of the water-gas separator 104 and predict whether flooding will occur at the anode 112 based on the anode inlet humidity and the separation efficiency. For example, if the humidity at the inlet 118 is too high, it may indicate that liquid water has accumulated at the anode 112. At this point, the control unit 122 may judge whether the water-gas separator 104 can capture the water in the gas at a normal level based on the separation efficiency of the water-gas separator 104. If the separation efficiency of the water gas separator 104 is insufficient to reach a normal level due to reasons such as aging, it can be inferred that flooding will occur at the anode 112 and corresponding protective measures can be taken to reduce the damage to the stack 102 caused by the flooding. In this way, the possibility of flooding at the anode can be determined in advance of the damage to the stack, thereby enabling timely application of appropriate protective measures to reduce the damage to the stack from the flooding.

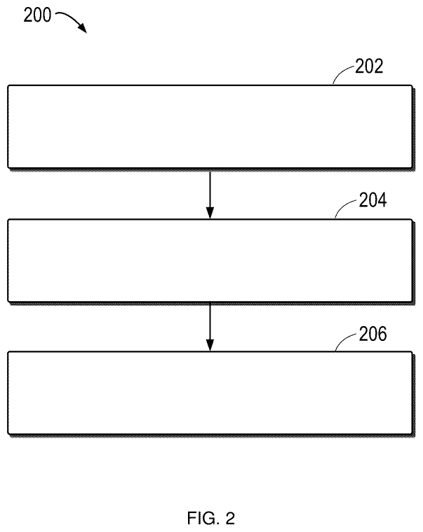

FIG. 2 shows a flow chart of a method 200 for determining the water condition at a fuel cell system anode according to some examples of the present disclosure. The method 200 may be performed, for example, by a control unit 122 in the environment 100. As shown in FIG. 2, at block 202, the method 200 may determine the anode inlet humidity of the fuel cell system. For example, in the environment 100 as shown in FIG. 1, the control unit 122 may determine the humidity at the inlet 118 of the anode 112 of the stack 102. In some examples, the humidity at the inlet 118 may be captured by a humidity sensor and communicated to the control unit 122. In some examples, the control unit 122 may collect operating parameters associated with the fuel cell system and calculate the humidity at the inlet 118 based on these operating parameters. In some examples, the control unit 122 may collect a data set including operating parameters associated with the fuel cell system and the humidity at the inlet 118 and utilize the data set to train a machine learning model for predicting anode inlet humidity. The control unit 122 may then utilize the trained machine learning model to infer the humidity at the inlet 118.

In block 204, the method 200 may determine the separation efficiency of the water-gas separator of the fuel cell system. For example, in the environment 100 as shown in FIG. 1, the control unit 122 may determine the separation efficiency of the water-gas separator 104. The water-gas separator 104 is used to separate water from the water-gas mixture discharged from the outlet 120 of the anode 112, while separation efficiency refers to the extent to which the water-gas separator 104 is able to effectively separate water and gas. The separation efficiency is generally expressed in percent; i.e., the ratio of the amount of water that the water-gas separator successfully separates to the total amount of water input. For example, a water-gas separator with 90% separation efficiency means that it is able to successfully separate 90% of the water, while the remaining 10% of the water will enter the rest of the fuel cell system, e.g., by circulating back to the anode 112 under the action of the hydrogen circulating pump 110.

In block 206, the method 200 may determine the condition of the water at the anode of the fuel cell system based on the anode inlet humidity and the separation efficiency of the water-gas separator. For example, in the environment 100 as shown in FIG. 1, the control unit 122 may predict that the anode 112 of the fuel cell system will be flooded based on the humidity at the inlet 118 of the anode 112 and the separation efficiency of the water-gas separator 104, allowing corresponding protective measures to be taken to reduce the damage to the stack 102 caused by the flooding. For example, the control unit 122 may determine whether liquid water has accumulated at the anode 112 based on the humidity at the inlet 118 of the anode 112 and then determine whether the water-gas separator 104 is still capable of supporting normal system operation based on the separation efficiency of the water-gas separator 104 to determine whether flooding will occur at the anode 112.

In this way, the possibility of flooding at the anode 112 can be determined in advance of the damage to the stack 102, thereby enabling timely application of appropriate protective measures to reduce the damage to the stack 102 from the flooding. In addition, using the humidity at the inlet 118 of the anode 112 and the separation efficiency of the water-gas separator 104 to predict flooding can reduce additional sensors and hardware equipment (such as CVMs), thereby reducing costs and improving system robustness.

In some examples, in order to determine whether flooding will occur at the anode, it may be determined that flooding will occur at the anode in response to the anode inlet humidity being greater than a first predetermined humidity threshold and the separation efficiency of the water-gas separator being less than a predetermined efficiency threshold. In some examples, in response to determining that flooding will occur at the anode, the stack of the fuel cell system may be shut down and an error message sent to provide a prompt via an interactive device of the vehicle that the anode will flood.

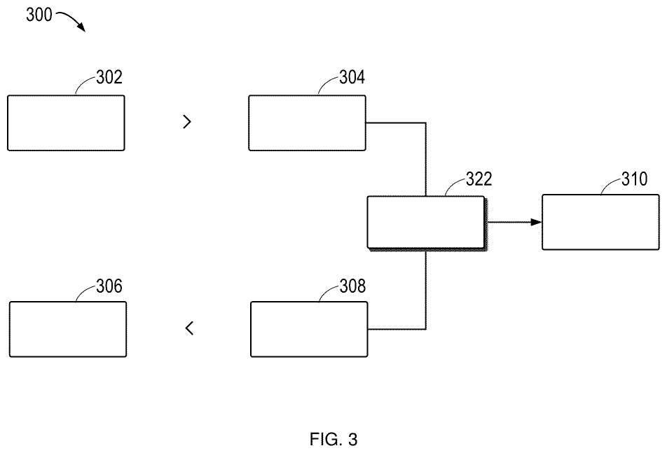

FIG. 3 shows a schematic diagram of an example 300 of predicting that flooding will occur at the anode based on anode inlet humidity and the separation efficiency of the water-gas separator according to some examples of the present disclosure. In example 300, control unit 322 (e.g., control unit 122 of the fuel cell system in FIG. 1) may determine the anode inlet humidity 302 (e.g., the humidity at the inlet 118 of the anode 112 in FIG. 1) and the separation efficiency 306 of the water-gas separator (e.g., the separation efficiency of the water-gas separator 104 in FIG. 1). The control unit 322 may then compare the anode inlet humidity 302 to a predetermined humidity threshold 304 (e.g., 90% or 95%) and compare the separation efficiency 306 of the water-gas separator to a predetermined efficiency threshold 308 (e.g., 80%, 85%, or 90%). If the anode inlet humidity 302 is greater than the predetermined humidity threshold 304, liquid water may have accumulated at the anode. At this point, if the separation efficiency 306 of the water-gas separator is less than the predetermined efficiency threshold 308, then this means that a larger portion of the water entering the water-gas separator cannot be successfully separated by the water-gas separator, and such water will circulate back to the anode of the stack under the action of the hydrogen circulating pump. At this point, the water can no longer be separated normally by the water-gas separator, which may lead to flooding at the anode. Accordingly, the control unit 322 may generate a flooding alarm 310 indicating that flooding will occur at the anode.

After generating the flooding alarm 310, the control unit 322 may shut down the stack to protect the stack from damage caused by the flooding. In addition, the control unit 322 of the fuel cell system may also send a message to the vehicle control unit that includes a flooding alarm 310. The vehicle control unit may then provide a prompt, based on the flooding alarm 310, that the anode will be flooded via an interactive device of the vehicle (such as a dash, media center, speaker, vibrating seat, etc.), which may be in the form of text, icons, speech, vibrations, etc. or a combination thereof.

In this way, an early warning of flooding can be given before the anode is flooded so that the stack can be shut down in time to avoid damage from flooding. Moreover, prompting the user with a flood alarm through the vehicle's interactive device may enable the user to make corresponding responses in time, improving the user experience.

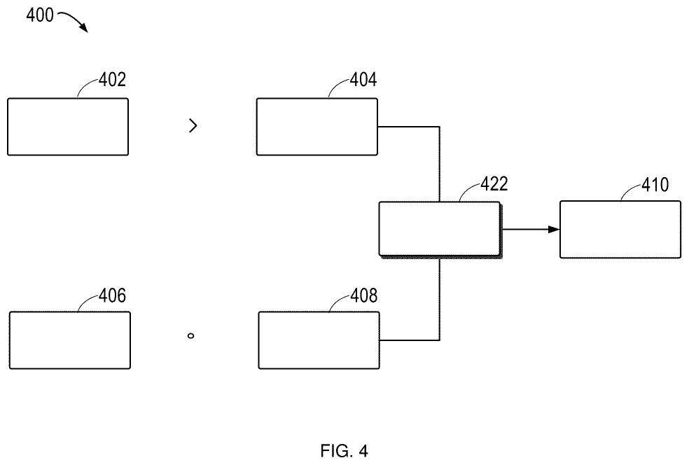

In some examples, it may be determined that no flooding has occurred at the anode in response to the anode inlet humidity being greater than a first predetermined humidity threshold and the separation efficiency of the water-gas separator not being less than a predetermined efficiency threshold. In some example, a drain valve associated with the water-gas separator may be opened in response to determining that no flooding has occurred at the anode. FIG. 4 shows a schematic diagram of an example 400 of controlling a drain valve based on anode inlet humidity and the separation efficiency of the water-gas separator according to some examples of the present disclosure. In example 400, the control unit 422 may determine the anode inlet humidity 402 and the separation efficiency 406 of the water-gas separator. The control unit 422 may then compare the anode inlet humidity 402 to the predetermined humidity threshold 404 (e.g., this could be the predetermined efficiency threshold 304 in FIG. 3) and the separation efficiency 406 of the water-gas separator to the predetermined efficiency threshold 408 (e.g., this could be the predetermined efficiency threshold 308 in FIG. 3).

As shown in FIG. 4, if the anode inlet humidity 402 is greater than the predetermined humidity threshold 404, liquid water may have accumulated at the anode. At this point, if the separation efficiency 406 of the water-gas separator is greater than or equal to the predetermined efficiency threshold 408, then the liquid water accumulated at the anode may be due to an inappropriate opening and closing strategy of the drain valve (e.g., the drain valve 106 in FIG. 1). If the drain valve is open less frequently or for a shorter period of time, it may not be possible to discharge the separated water from the water-gas separator out of the fuel cell system in a timely manner, resulting in the water in the water-gas separator being recycled back to the anode of the stack, which causes the anode to be flooded. Accordingly, in this instance, control unit 422 may send an opening instruction 410 to the drain valve to cause the drain valve to drain the water accumulated in the water-gas separator from the fuel cell system. In some examples, the control unit 422 may also increase the opening frequency of the drain valve or extend the duration of time the drain valve is opened to reduce liquid water accumulated at the anode.

In this way, the drain valve can be automatically adjusted when the anode is likely to be flooded, thereby effectively reducing the risk of anode flooding, improving the safety of the fuel cell system, and extending the life of the stack. In addition, the frequency at which the power stack shuts down as a result of predicted flooding can also be reduced, thereby increasing the efficiency of the fuel cell system.

In some examples, to further improve the accuracy of the prediction of flooding at the anode, the high-frequency impedance of the stack may be additionally be considered. In some examples, the high-frequency impedance of the stack of the fuel cell system may be determined prior to determining the anode inlet humidity of the fuel cell system, and the water condition at the anode of the fuel cell system may be determined based on the high-frequency impedance of the stack. In some examples, before determining the anode inlet humidity of the fuel cell system, it may be determined water flooding has occurred at the anode or whether the anode is membrane-dry based on the high-frequency impedance of the stack. In some examples, in response to the high-frequency impedance of the stack being less than a first predetermined impedance threshold, indicating the possibility of flooding at the anode, the anode inlet humidity and the separation efficiency of the water-gas separator may further be determined and the water condition at the anode may be determined based on the anode inlet humidity, the separation efficiency of the water-gas separator, and the high-frequency impedance. In some examples, in response to the anode inlet humidity being greater than a first predetermined humidity threshold, the separation efficiency of the water-gas separator being less than a predetermined efficiency threshold, and the high-frequency impedance of the stack being less than a first predetermined impedance threshold, it is determined that flooding will occur at the anode.

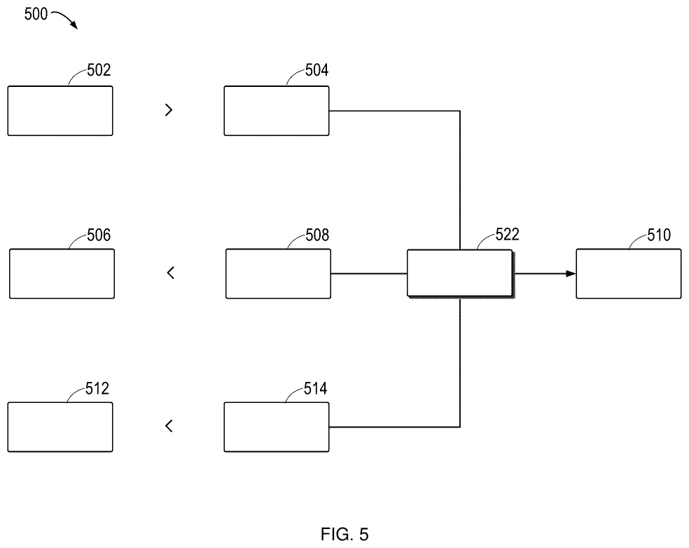

FIG. 5 shows a schematic diagram of an example 500 of using the high-frequency impedance of the stack to verify whether liquid water has accumulated at the anode according to examples of the present disclosure. FIG. 5 shows a schematic diagram of an example 500 of predicting that flooding will occur at the anode based on anode inlet humidity, the separation efficiency of the water-gas separator, and the high-frequency impedance of the stack according to some examples of the present disclosure. In example 500, control unit 522 may determine the anode inlet humidity 502, the separation efficiency 506 of the water-gas separator, and the high-frequency impedance 512 of the stack (e.g., the stack 102 in FIG. 1). In some examples, electrochemical impedance spectroscopy (EIS) may be utilized to determine the high-frequency impedance 512 of the stack. A DC-DC converter may send a sinusoidal current signal to the stack and receive a voltage signal back from the stack, based on which the high-frequency impedance 512 of the stack may be determined. In a stack, the protons bind to and transmit through water. Thus, the more water at the proton exchange membrane (e.g., the proton exchange membrane 116 in the FIG. 1), the more protons are transferred and the smaller the high-frequency impedance of the stack. Conversely, if there is less water at the proton exchange membrane of the stack, the number of protons that can be transmitted will be smaller and the high-frequency impedance of the stack will be greater.

In example 500, the control unit 522 of the fuel cell system may compare the anode inlet humidity 502 to a predetermined humidity threshold 504 (e.g., the predetermined humidity threshold 304 in FIG. 3), the separation efficiency 506 of the water-gas separator to a predetermined efficiency threshold 508 (e.g., the predetermined efficiency threshold 308 in FIG. 3), and the high-frequency impedance 512 of the stack to a predetermined impedance threshold 514. If the control unit 522 determines that the anode inlet humidity 502 is greater than the predetermined humidity threshold 504, liquid water may have accumulated at the anode. At this point, if the high-frequency impedance 512 is less than the predetermined impedance threshold 514, then the high-frequency impedance 512 is smaller, which may indicate more water at the proton exchange membrane. In these examples, the humidity status of the stack indicated by the anode inlet humidity 502 and the high-frequency impedance 512 of the stack is consistent; that is, both indicate that liquid water may have accumulated at the anode, thereby verifying that liquid water may have accumulated at the anode. Further, if the separation efficiency 506 of the water-gas separator is less than the predetermined efficiency threshold 508, this means that the water-gas separator has not been able to properly separate the water, which may result in flooding at the anode. Accordingly, the control unit 522 may generate a flooding alarm 510 indicating that flooding will occur at the anode.

In some examples, in response to the high-frequency impedance of the stack not being less than the first predetermined impedance threshold and not being greater than the second predetermined impedance threshold, the anode is determined to have not been flooded, or in response to the high-frequency impedance of the stack being greater than the second predetermined impedance threshold, the stack is determined to be membrane-dry. For example, when the high-frequency impedance of the stack is not greater than a larger impedance threshold, this indicates that there is a certain amount of liquid water at the proton exchange membrane, which can carry a corresponding number of protons, causing the impedance of the stack to be no greater than a larger impedance threshold. Therefore, it can at least be determined that no flooding has occurred at the anode. If it is desired to further determine whether the anode is membrane-dry, it may be determined whether the high-frequency impedance of the stack is greater than a larger impedance threshold. When the high-frequency impedance of the stack is greater than a larger impedance threshold, this indicates that the amount of liquid water at the proton exchange membrane is too low, resulting in too few protons being transferred. Thus, it can be determined that the stack is membrane-dry.

In some examples, where both anode inlet humidity and the high-frequency impedance of the stack are determined, in response to the anode inlet humidity being greater than a first predetermined humidity threshold and the high-frequency impedance of the stack being greater than a second predetermined impedance threshold, it is determined that the anode has not been flooded. In these examples, if it has been determined that the anode has not been flooded, there is no need to determine the separation efficiency of the water-gas separator and determine whether the anode will be flooded based on the humidity at the anode inlet and the separation efficiency of the water-gas separator.

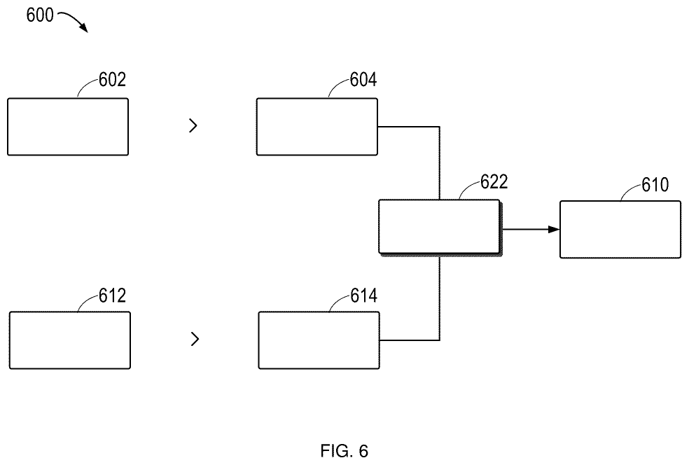

FIG. 6 shows a schematic diagram of an example 600 in which the state of a stack as indicated by the anode inlet humidity and the high-frequency impedance of the stack is inconsistent according to some examples of the present disclosure. In example 600, the control unit 622 may determine the anode inlet humidity 602 and the high-frequency impedance 612 of the stack. The control unit 622 may then compare the anode inlet humidity 602 to the predetermined humidity threshold 604 and compare the high-frequency impedance 612 of the stack to the predetermined impedance threshold 614. If the control unit 622 determines that the anode inlet humidity 602 is greater than the predetermined humidity threshold 604, liquid water may have accumulated at the anode. At this point, if the high-frequency impedance 612 is greater than the predetermined impedance threshold 614, then the high-frequency impedance 612 is relatively large, which may indicate that the proton exchange membrane is dry. At this point, the humidity state of the stack as indicated by the anode inlet humidity 602 and the high-frequency impedance 612 of the stack is inconsistent. Thus, the control unit 622 can generate a conflict alarm 610 indicating that the humidity state of the stack as indicated by the anode inlet humidity 602 and the high-frequency impedance 612 of the stack is inconsistent. The conflict alert 610 may be displayed to the user or uploaded to the cloud for engineer analysis. It should be noted that the predetermined impedance threshold 614 may be the same value as the predetermined impedance threshold 514 in FIG. 5 or may be a different value. In some examples, the predetermined impedance threshold 614 may be greater than the predetermined impedance threshold 514 in FIG. 5.

In this way, the control unit 622 can determine the humidity state of the stack by combining the high-frequency impedance of the stack and the anode inlet humidity, thereby increasing the accuracy of the prediction of the anode flooding, reducing erroneous shutdowns of the stack, and displaying flooding alarms to the user, which can improve the efficiency of the fuel cell system and the user experience.

In some examples, in order to further improve the accuracy of judging whether the stack is membrane-dry, whether the stack is membrane-dry may be determined based on both the anode inlet humidity and the high-frequency impedance of the stack so that water conservation measures can be taken in time. In some examples, in response to the anode inlet humidity being less than a second predetermined humidity threshold and the high-frequency impedance of the stack being greater than a second predetermined impedance threshold, it is determined that the stack is membrane-dry. In these examples, if it has been determined that the stack is membrane-dry, there is no need to determine the separation efficiency of the water-gas separator and determine whether the anode will be flooded based on the humidity at the anode inlet and the separation efficiency of the water-gas separator. In some examples, in response to determining that the stack is membrane-dry, at least one of the following measures may be performed: reducing the amount of cathode air, decreasing the coolant temperature, or increasing the cathode back pressure of the stack.

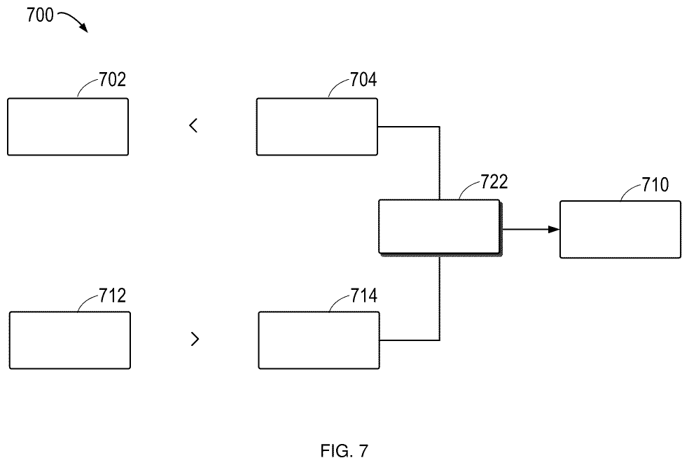

FIG. 7 shows a schematic diagram of an example 700 of determining a stack is membrane-dry based on the anode inlet humidity and the high-frequency impedance of the stack according to some examples of the present disclosure. In example 700, the control unit 722 may determine the anode inlet humidity 702 and the high-frequency impedance 712 of the stack. The control unit 722 may then compare the anode inlet humidity 702 to the predetermined humidity threshold 704 and compare the high-frequency impedance 712 of the stack to the predetermined impedance threshold 714. If the anode inlet humidity 702 is less than the predetermined humidity threshold 704, then the amount of liquid water accumulated at the anode is too low. If the high-frequency impedance 712 of the stack is greater than the predetermined impedance threshold 714, this means that the amount of water in the stack is too low. At this time, the anode inlet humidity 702 and the high-frequency impedance 712 of the stack both indicate that the amount of water in the stack is too low. The amount of water in the stack being too low is referred to as membrane dryness, which reduces the conductivity of the proton exchange membrane and will cause irreversible membrane damage when severe. Accordingly, the control unit 722 may generate a membrane dryness alert 710, and the fuel cell system may then take a series of water conservation measures on the stack based on the membrane dryness alert 710.

As air enters the stack from the cathode (e.g., the cathode 114 in FIG. 1), the greater the amount of air and the greater the airflow, the more moisture is carried away. As such, the fuel cell system may reduce the amount of air in the stack cathode, thereby reducing the amount of moisture carried away by the airflow and serving to conserve water. Moreover, as the coolant can flow through the stack, the higher the temperature of the coolant, the more water is evaporated and the more water is carried away by the airflow. As such, the fuel cell system can reduce the temperature of the coolant, thereby reducing the evaporation of water and serving to conserve water. In addition, increasing the back pressure at the stack cathode outlet may also reduce the loss of water in the stack. For example, the control unit 722 may increase the back pressure at the cathode outlet by closing a back pressure valve connected to the cathode outlet. By combining different water conservation measures, it is possible to reduce the side effects of applying a single water conservation measure alone while improving the effect of water conservation.

In this way, whether the stack is membrane-dry can be effectively determined and the accuracy of the diagnosis of membrane dryness can be improved by utilizing the anode inlet humidity 702 and the high-frequency impedance 712 of the stack for mutual verification. In addition, water conservation measures help maintain proper working conditions for the proton exchange membrane, improving the performance, stability, and life of the fuel cell system.

In some examples, for purposes of determining anode inlet humidity, the anode inlet temperature, the anode inlet pressure, the hydrogen injector inlet temperature, the hydrogen injector inlet pressure, and the stack current can be determined, and the anode inlet humidity is determined based on the anode inlet temperature, the anode inlet pressure, the hydrogen injector inlet temperature, the hydrogen injector inlet pressure, and the stack current.

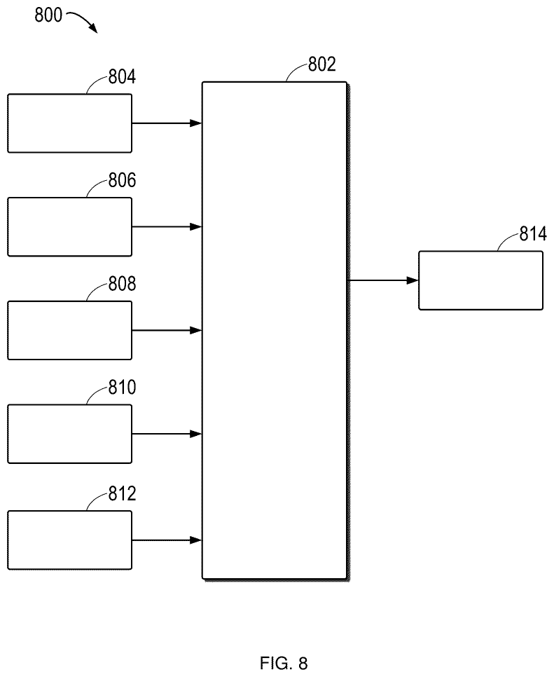

FIG. 8 shows a schematic diagram of an example 800 of using a virtual humidity sensor to determine anode inlet humidity according to some examples of the present disclosure. As shown in FIG. 8, the example 800 includes a virtual humidity sensor 802 that is a software model for determining anode inlet humidity, and the virtual humidity sensor 802 may be implemented by the control unit of the fuel cell system (e.g., control unit 122 in FIG. 1). The fuel cell system control unit may determine the anode inlet temperature 804, the anode inlet pressure 806, the hydrogen injector inlet temperature 808, the hydrogen injector inlet pressure 810, and the stack current 812. The data may then be input to the virtual humidity sensor 802 and the virtual humidity sensor 802 determines and outputs the anode inlet humidity 814 based on these input data.

In some examples, the virtual humidity sensor 802 may utilize a multilayer perceptron and generate the anode inlet humidity 814 based on the anode inlet temperature 804, the anode inlet pressure 806, the hydrogen injector inlet temperature 808, the hydrogen injector inlet pressure 810, and the stack current 812. In these examples, the virtual humidity sensor 802 may pre-process these data. For example, the virtual humidity sensor 802 may use methods such as filling, deletion, or interpolation to handle missing values in the data. The virtual humidity sensor 802 may then detect and process outliers in the data and standardize the data. This may harmonize the input data of the multilayer perceptron to a similar range of values, thereby ensuring the stability and accuracy of the multilayer perceptron.

The multilayer perceptron of the virtual humidity sensor 802 may include an input layer, a plurality of hidden layers, and an output layer, wherein the input layer includes five nodes that correspond to the anode inlet temperature 804, the anode inlet pressure 806, the hydrogen injector inlet temperature 808, the hydrogen injector inlet pressure 810, and the stack current 812, respectively. Each hidden layer of the multilayer perceptron has a plurality of nodes (e.g., 16 nodes, 8 nodes, or more or fewer nodes), and the plurality of nodes of each layer are connected to the plurality of nodes of the first layer. Each node of the first hidden layer of the plurality of hidden layers is connected to each node of the input layer, and the multilayer perceptron may use a rectified linear unit (ReLU) function as an activation function of the hidden layer. The output layer of the multilayer perceptron has a node corresponding to the anode inlet humidity 814. The multilayer perceptron may use the linear activation function as the activation function of the output layer to output the anode inlet humidity 814.

In this way, the virtual humidity sensor 802 can be implemented by software, enabling the determination of humidity at the anode inlet, which not only saves hardware costs but also improves system stability without concerns about the aging and service life of hardware sensors.

In some examples, for purposes of determining the separation efficiency of the water-gas separator, the water-gas separator inlet pressure, the water-gas separator outlet pressure, the stack current, the anode inlet pressure, the cathode outlet pressure, and the stack aging degree may be determined. The separation efficiency of the water-gas separator may then be determined based on the water-gas separator inlet pressure, the water-gas separator outlet pressure, the stack current, the anode inlet pressure, the cathode outlet pressure, and the stack aging degree.

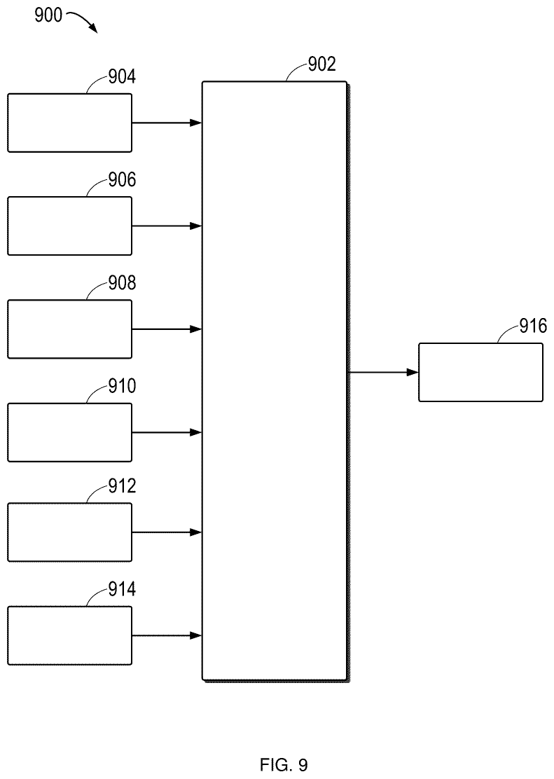

FIG. 9 shows a schematic diagram 900 of an example of using a separation efficiency determination model to determine the separation efficiency of the water-gas separator according to some examples of the present disclosure. As shown in FIG. 9, the example 900 includes a separation efficiency determination model 902, and the separation efficiency determination model 902 may be implemented by the control unit of the fuel cell system (e.g., control unit 122 in FIG. 1). The fuel cell system control unit may determine the water-gas separator inlet pressure 904, the water-gas outlet pressure 906, the stack current 908, the anode inlet pressure 910, the cathode outlet pressure 912, and the stack aging degree 914. The data may then be input into the separation efficiency determination model 902 and the separation efficiency 916 of the water-gas separator is determined based on these input data by the separation efficiency determination model 902. The separation efficiency 916 may be defined as the ratio of the water separated by the water-gas separator to the water entering the water-gas separator. The differential pressure between the anode inlet pressure 910 and the cathode outlet pressure 912 and the stack current 908 may reflect the amount of water entering the water-gas separator, e.g., the greater the pressure differential or the greater the stack current, the greater the amount of water entering the water-gas separator. The differential pressure between the water-gas separator inlet pressure 904 and the water-gas separator outlet pressure 906 may reflect the amount of water separated by the water-gas separator, e.g., the greater the differential pressure, the greater the amount of water separated by the water-gas separator. Further, if the stack aging degree 914 can also reflect the amount of water entering the water-gas separator, for example, the higher the stack aging degree, the more water penetrates from the cathode to the anode and the greater the amount of water entering the water-gas separator.

By considering the parameters associated with the water-gas separator together with the stack aging degree, the separation efficiency of the water-gas separator can be calculated more precisely, thereby increasing the accuracy of determining the separation efficiency of the water-gas separator. In addition, improving the accuracy of the separation efficiency of the water-gas separator can also improve the accuracy of the prediction of flooding at the anode, thereby reducing false and missed alarms of flooding and improving the stability and robustness of the fuel cell system.

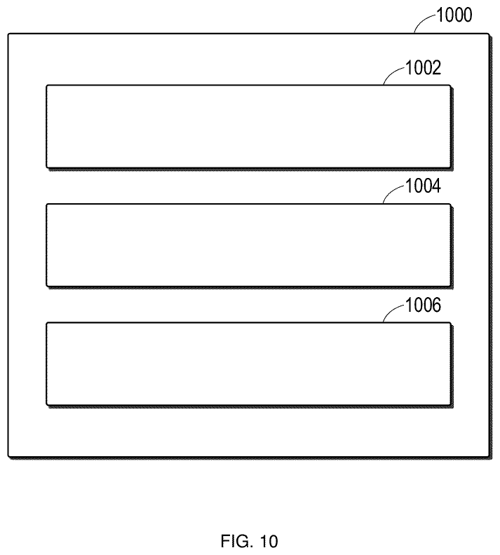

FIG. 10 shows a block diagram of a device 1000 for determining the water condition at a fuel cell system anode according to some examples of the present disclosure. As shown in FIG. 10, the device 1000 includes a humidity determination unit 1002 configured to determine the anode inlet humidity of a fuel cell system. The device 1000 further comprises a separation efficiency determination unit 1004 configured to determine the separation efficiency of the water-gas separator of the fuel cell system. Additionally, the device 1000 further comprises a flooding monitoring unit 1006 configured to determine the condition of the water at the anode of the fuel cell system based on the anode inlet humidity and the separation efficiency of the water-gas separator.

It should be understood that by utilizing the device 1000 of the present disclosure, at least one of a number of advantages that are capable of being implemented by the method or process as described above can be implemented. For example, the device 1000 can determine the possibility of flooding at the anode in advance of the damage to the stack, thereby enabling timely application of appropriate protective measures to reduce the damage to the stack from the flooding.

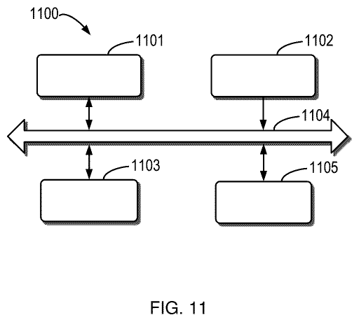

FIG. 11 shows a block diagram of a controller 1100 that can implement a plurality of examples of the present disclosure. The controller 1100, for example, may be the control unit 122 of the fuel cell system as shown in FIG. 1. As shown in the figure, the controller 1100 includes a processor 1101, which can perform various appropriate actions and processes according to computer program instructions stored in a read-only memory (ROM) 1102 and loaded into a random-access memory (RAM) 1103. Various programs and data required for the operation of the controller 1100 may also be stored in the RAM 1103. The processor 1101, the ROM 1102, and the RAM 1103 are interconnected through a bus 1104. An input/output (I/O) interface 1105 is also connected to the bus 1104.

The processor 1101 can be various general-purpose and/or special-purpose processing components with processing and computing capabilities. Examples of the processor 1101 include, but are not limited to, central processing units (CPU), graphics processing units (GPU), various dedicated artificial intelligence (AI) computing chips, various computing units running machine learning model algorithms, digital signal processors (DSP), and any appropriate processors, controllers, microcontrollers, etc. The processor 1101 performs various methods and processes described above, such as the method 200. For example, in some examples, the method 200 can be implemented as a computer software program tangibly contained in a machine-readable medium. In some examples, part or all of the computer programs may be loaded and/or installed onto the controller 1100 through the ROM 1102. When the computer program is loaded into the RAM 1103 and executed by the processor 1101, one or more steps of the method 200 described above can be performed. Alternatively, in other examples, the processor 1101 can be configured to perform method 200 by any other suitable means (e.g., by means of firmware).

The functions described above herein can be performed, at least in part, by one or more hardware logic components. For example, and without limitation, exemplary types of hardware logic components that can be used include: Field Programmable Gate Arrays (FPGA), Application Specific Integrated Circuits (ASIC), Application Specific Standard Products (ASSP), System on a Chip (SOC), Complex Programmable Logic Devices (CPLD), and the like.

The program code for implementing the methods of the present disclosure can be written in any combination of one or more programming languages. This program code can be provided to a processor or controller of a general-purpose computer, special-purpose computer, or other programmable data processing apparatus such that the program code, when executed by the processor or controller, causes the functions/operations specified in the flowcharts and/or block diagrams to be implemented. The program code can be executed entirely on the machine, partly on the machine, as a stand-alone software package partly on the machine and partly on a remote machine, or entirely on a remote machine or server.

In the context of the present disclosure, a machine-readable medium can be a tangible medium that can contain or store programs for use by or in conjunction with an instruction execution system, apparatus, or device. A machine-readable medium can be a machine-readable signal medium or a machine-readable storage medium. The machine-readable medium can include, but is not limited to, electronic, magnetic, optical, electromagnetic, infrared, or semiconductor systems, apparatuses, or devices, or any suitable combination of the foregoing. More specific examples of the machine-readable storage medium would include electrical connections based on one or more wires, portable computer disks, hard disks, random access memory (RAM), read-only memory (ROM), erasable programmable read-only memory (EPROM or Flash memory), optical fibers, portable compact disc read-only memory (CD-ROM), optical storage devices, magnetic storage devices, or any suitable combination of the foregoing. Furthermore, although operations have been depicted in a specific order, it should be understood that such operations are not required to be performed in the specific order shown or in sequential order, nor are all illustrated operations required to be performed to achieve the desired results. In certain contexts, multitasking and parallel processing may be advantageous. Similarly, although several specific implementation details are included in the above discussion, these should not be construed as limiting the scope of the present disclosure. Certain features described in the context of separate examples can also be implemented in combination in a single implementation. Conversely, various features described in the context of a single implementation can also be implemented separately or in any suitable sub-combination in multiple implementations.

Although the subject matter has been described in language specific to structural features and/or methodological acts, it is to be understood that the subject matter defined in the appended claims is not necessarily limited to the specific features or acts described above. Rather, the specific features and operations described above are merely exemplary forms of implementing the claims.

What is claimed is:

1. A method for determining condition of water at an anode of a fuel cell system, comprising:
determining an anode inlet humidity of the fuel cell system;
determining a separation efficiency of a water-gas separator of the fuel cell system; and
determining the condition of the water at the anode of the fuel cell system based on the anode inlet humidity and the separation efficiency of the water-gas separator.

2. The method according to claim 1, wherein determining the condition of the water at the anode of the fuel cell system based on the anode inlet humidity and the separation efficiency of the water-gas separator comprises at least one of:
in response to the anode inlet humidity being greater than a first predetermined humidity threshold and the separation efficiency of the water-gas separator being less than a predetermined efficiency threshold, determining that flooding will occur at the anode; and
in response to the anode inlet humidity being greater than the first predetermined humidity threshold and the separation efficiency of the water-gas separator being no less than a predetermined efficiency threshold, determining that no flooding has occurred at the anode.

3. The method according to claim 2, further comprising: in response to determining that flooding will occur at the anode:
shutting down a stack of the fuel cell system, and
sending an error message to provide a prompt that flooding will occur at the anode via an interactive device of a vehicle.

4. The method according to claim 2, further comprising:
opening a drain valve associated with the water-gas separator in response to determining that no flooding has occurred at the anode.

5. The method according to claim 1, further comprising: prior to determining the anode inlet humidity of the fuel cell system:
determining a high-frequency impedance of a stack of the fuel cell system; and
determining the condition of the water at the anode of the fuel cell system based on the high-frequency impedance of the stack.

6. The method according to claim 5, wherein determining the condition of the water at the anode of the fuel cell system based on the high-frequency impedance of the stack comprises at least one of:
determining that flooding has not occurred at the anode in response to the high-frequency impedance of the stack being not less than a first predetermined impedance threshold and not greater than a second predetermined impedance threshold; and
determining that the stack is membrane-dry in response to the high-frequency impedance of the stack being greater than the second predetermined impedance threshold.

7. The method according to claim 6, wherein determining the anode inlet humidity of the fuel cell system comprises:
determining the anode inlet humidity of the fuel cell system in response to the high-frequency impedance of the stack being less than the first predetermined impedance threshold.

8. The method according to claim 6, further comprising:
in response to determining that the stack is membrane-dry, performing at least one of the following: reducing an amount of cathode air, decreasing a coolant temperature, or increasing a cathode back pressure of the stack.

9. The method according to claim 1, wherein determining the anode inlet humidity of the fuel cell system comprises:
determining an anode inlet temperature, an anode inlet pressure, a hydrogen injector inlet temperature, a hydrogen injector inlet pressure, and a stack current; and
determining the anode inlet humidity based on the anode inlet temperature, the anode inlet pressure, the hydrogen injector inlet temperature, the hydrogen injector inlet pressure, and the stack current.

10. The method according to claim 1, wherein determining the separation efficiency of the water-gas separator of the fuel cell system comprises:
determining an inlet pressure and an outlet pressure of the water gas separator, a stack current, an anode inlet pressure, a cathode outlet pressure, and a stack aging degree; and
determining the separation efficiency of the water-gas separator based on the inlet pressure and the outlet pressure of the water-gas separator, the stack current, the anode inlet pressure, the cathode outlet pressure, and the stack aging degree.

11. A device for determining condition of water at an anode of a fuel cell system, comprising:
a humidity determination unit configured to determine an anode inlet humidity of the fuel cell system;
a separation efficiency determination unit configured to determine a separation efficiency of a water-gas separator of the fuel cell system; and
a flooding monitoring unit configured to determine the condition of the water at the anode of the fuel cell system based on the anode inlet humidity and the separation efficiency of the water-gas separator.

12. A controller, comprising:
at least one processor; and
a memory, coupled to the at least one processor, and having instructions stored thereon that, when executed by the at least one processor, cause the controller to perform the method according to claim 1.

13. A vehicle, comprising the controller according to claim 12.

14. A machine-readable storage medium storing machine-executable instructions, wherein the machine-executable instructions are executed by a processor to implement the method according to claim 1.
